(19) United States
(12) Patent Application Publication
(10) Pub. No.: US 2020/0306057 A1
(43) Pub. Date: Oct. 1, 2020

(54) MODULAR PROSTHETIC ARM SYSTEM

(71) Applicant: PROYECTOS OFTALMOLOGICOS, S.A. DE C.V., Culiacan De Rosales (MX)

(72) Inventors: Adrian Armenta Meza, Culiacan (MX); Oscar Esteban Cazares Almaral, Culiacan (MX); Rafael Irving Esparza Gutierrez, Culiacan (MX); Jesus Manuel Herrera Hernandez, Culiacan (MX)

(21) Appl. No.: 16/759,650

(22) PCT Filed: Oct. 26, 2018
(86) PCT No.: PCT/MX2018/000112

§ 371 Date: Apr. 27, 2020

(30) Foreign Application Priority Data
Oct. 27, 2017 (MX) MX/A/2017/013912

(51) Int. Cl. A61F 2/58; A61F 2/78; A61F 2/80; A61F 2/72

(52) U.S. Cl. CPC A61F 2/586; A61F 2/7843; A61F 2002/543; A61F 2/72; A61F 2/80

(57) ABSTRACT

Modular arm prosthesis, for amputations higher than the level of the forearm. With the feature of being modular, which gives us an advantage that reduces the repair time by replacing the module quickly. The Socket-stump module which can be adjusted to several stumps thanks to its pneumatic system for grip, decreases the level of customization of each prosthesis thus reducing the manufacturing time and cost of production of each prosthesis, as well as its easy control by means of myoelectric sensors.

### TECHNICAL FIELD

The present invention relates to the field of prosthesis, specifically to modular prosthesis for people who lack the member at the transradial level.

### BACKGROUND OF THE INVENTION

Prior art in document US 2005/0021154 A1 describes a handling device for a finger prosthesis comprising a method and apparatus for determining the complex impedance of an electrical component. The method comprises the steps of applying an input signal to the component comprising a plurality of discrete frequencies simultaneously and determining the complex impedance of the component at each of the frequencies using a discrete demodulation technique in two parameters related to complex impedance at each of the discrete frequency values. This method is applicable in electrically noisy environments and can be used to determine the impedance and equivalent circuit parameters for a battery. It can also be applied to battery system interconnects to allow battery system currents to be determined. U.S. Pat. No. 4,094,016 is also available as part of the state of the art, which describes an artificial hand and forearm comprising an artificial hand and a forearm comprise an elongated open housing at its rear end adapted to receive the piece of a human forearm and adjacent arm, adapted for fixing. A plate inside the housing mounted pivotally a series of finger sets inside the housing, with such finger sets sticking out of the housing. A thumb set is separated from such finger sets and mounted pivotally on the plate, with the thumb assembly sticking out of the housing and opposite the index finger set. A camshaft is articulated on that plate and mounts a series of spaced cams that are recorded respectively with the inner ends of the finger sets. A cam includes a pair of opposite cam surfaces for simultaneous recording with the index finger and thumb sets. A spring presses such finger sets in an open position against these cams. A reversible electric motor inside the housing has an output shaft engaged to the camshaft. A power supply, switch, and electrical circuit inside the housing connect the power supply. The switch under the control of a piece of arm can be activated in one direction, turning those cams in a direction that moves those sets of finger and thumb inwards to grab an object, disabling that switch by stopping those fingers. Activate the switch successively in the opposite direction by reversing the motor, turning those cams in the opposite direction by turning those thumb and finger sets to move out by releasing that object.

Similarly, U.S. Pat. No. 4,246,661 is documented, describing a digitally controlled artificial hand that has a fully articulated wrist movement. The invention provides a digitally controlled remote manipulator device, capable of reproducing all main human forearm, wrist and hand movements in a compact online package. This movement is carried out without the feedback requirement, thus making the appropriate invention for digital computer control. The packaging of the device allows it to be sealed, which means that it can be used as a remote manipulator that can reproduce the human arm and hand movements. The package also produces a lightweight and cosmetically pleasing prosthetic device for human use.

Finally, point out to the MX 329845 document describing an adjustable socket for lower limb prosthesis which allows to adjust a prosthesis to variations, comprising: an adjustment system that compensates for the loss of muscle mass, a structure system that is a rigid support that provides support to the socket, a clamp, a support structure and a base system containing the inflate system of the fasteners and a bolt system hole that allows assembly of the adjustment system and the structure system.

The disadvantages presented by the other known prosthesis with respect to the invention are:

The prosthetic models on the market use their construction in one piece, where it is usually necessary to replace the entire device if any failure occurs. The prosthesis model of the present invention employs a modular construction, this allows to replace with independent modules if any of the fault is presented or if it is necessary to update them.

They have limited functions: Many of the prosthesis on the market have the capacity for a single movement, flex and extension, are totally mechanical or even do not have any movement. The prosthesis of the present invention allows independent movement in the thumb and forefinger fingers, so they can perform more precise functions for different activities.

They need to be customized: Normally the use of an arm prosthesis is conditioned to make it tailored to the user stump, in order to give safety and comfort in handling. This can present itself as a problem, because over time the user's dimensions can change and that means that the prosthesis must be redesigned. In addition, this design can take weeks or even months of development.

Its price is very high: when using conventional manufacturing methods such as CNC machining or plastic injection with molds, the final price of the prosthesis is increased, which leaves them at a disadvantage to our model of up to 20 times lower price.

The present invention is designed to help patients who suffer from the lack of some upper limb with forearm level, either for congenital reasons or by amputation, to their social and personal development. As for its functionality, it allows to execute day-to-day actions, such as drinking a glass, holding a pen, holding a card, holding a door, among others. It also implements a system adjustable to the shape of the user's stump, allowing the grip of the prosthesis to the body to be safe, comfortable and that, unlike other prosthesis on the market, does not need a specialist and weeks of suitability and training to be able to start using it.

It has a control system manipulated by myoelectric sensors, (not illustrated) which send signals obtained from the user's muscles and by processing in the control algorithm, executes the functions programmed in the prosthesis in a very intuitive and easy to learn way.

The arm prosthesis is made of quality materials and very durable, using latest generation manufacturing techniques that allow us to create complex parts at a low cost. This allows us to have a much lower price of the device than is handled in the market of arm prosthesis with similar functions, reaching up to 20 times lower price.

### SUMMARY OF THE INVENTION

The present invention provides a modular arm prosthesis, comprising: a first module that includes a motor system and a mechanism for finger movement and a control board; a second module that has a first side configured to attach to the first module and a second side configured to attach to a user's stump; the second module includes, inside, a pneumatic system with an air intake valve to inflate an inner part of the second module and an air ejection valve to deflate the inner part of the second module.

In one mode, the modular arm prosthesis comprises an intermediate module between the first module and the second module, the intermediate module includes a power supply battery for the prosthesis, said battery supplies power to the control board.

In another embodiment the modular arm prosthesis comprises a power supply battery in the first module for the prosthesis, said battery supplies power to a control board located in the first module.

Also, the present invention provides a module for modular arm prosthesis, comprising a first side configured to attach to an additional module and a second side configured to attach to a stump of a user; the module includes, inside, a pneumatic system with an air intake valve to inflate an inner part of the module and an air ejection valve to deflate the inner part of the module.

In a modular arm prosthesis module mode also comprises an intermediate module between the module and the additional module, the additional module includes a motor system and a finger movement mechanism and a control board, the intermediate module includes a power supply battery for the prosthesis, said battery supplies power to the control board.

Also, the module for modular arm prothesis includes a power supply battery in the additional module for the prosthesis, said battery supplies power to a control board located in the additional module.

### BRIEF DESCRIPTION OF THE DRAWINGS

For a further understanding of the present invention, reference should be had to the following detailed description, taken in conjunction with the accompanying drawings, and wherein:

FIG. 1 is a view of the arm prosthesis divided into modules of the present invention;

FIG. 2 is an exploded view of the hand palm module of the arm prosthesis of the present invention;

FIG. 3 is an exploded view of the Socket-Chassis module of the arm prosthesis of the present invention;

FIG. 4 is an exploded view of the Socket-stump module of the arm prosthesis of the present invention;

FIG. 5 is a sectional view of the assembly of the arm prosthesis of the present invention;

FIG. 6 is a sectional view of the socket-stump module assembly;

FIG. 7 is an blocks diagram of electrical connection, data reading and sending of control board signals for the operation of the modular prosthesis;

FIG. 8 is a flowchart of the modular prosthesis control algorithm of robotic arm, which explains how the functions of the prosthesis are controlled using 2 myoelectric sensors;

FIG. 9 is a view of the arm prosthesis divided into modules of an embodiment of the present invention; and

FIG. 10 is an exploited view of the hand palm module of the arm prosthesis of the embodiment of the present invention.

### DETAILED DESCRIPTION OF THE INVENTION

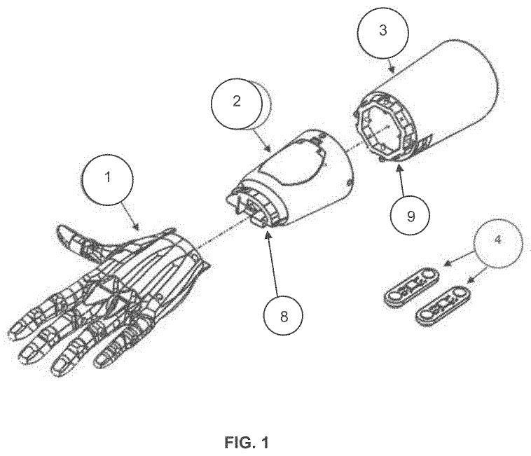

With reference to FIG. 1, you can see the different parts/modules of the prosthesis with their respective myoelectric sensors (1, 2, 3, 4). The arm prosthesis is a device for people who lack the limb at the transradial level, either due to congenital causes or amputations. The prosthesis consists of three main modules (1, 2, 3), the first module called hand palm (1) is responsible for storing the drive system, the control board and the mechanism responsible for the movement of the fingers, the second module called Socket-Chassis (2) has the function of being the coupling between the Hand palm and the Socket-stump (3), as well as the storage of the battery, and the third module called Socket-stump (3) which is responsible for attaching the prosthesis to the end user, which are connected to each other by means of assembly guides (8,9) which allow a firm and secure assembly.

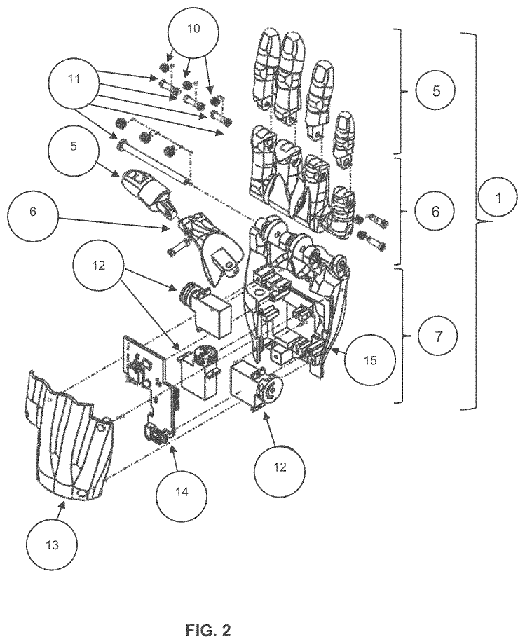

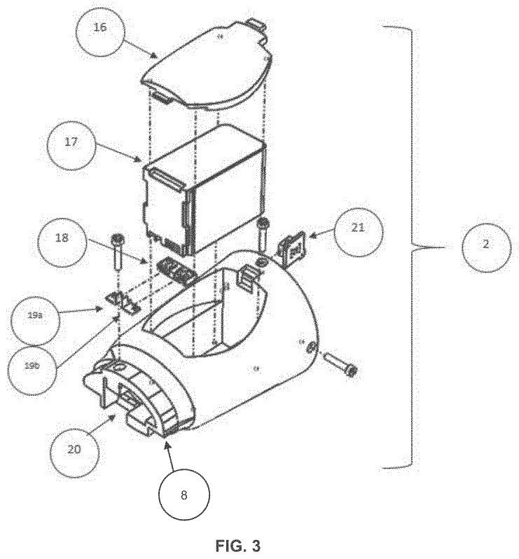

In relation to FIG. 2, the hand palm module (1) comprises distal fingers (5) and proximal fingers (6) which are the main part for gripping objects by duplicating the basic functions of a hand that are bending, extension, click and clamp. For the realization of these movements is used the drive system (7) responsible for generating the gripping force by means of servo motors (12), which are controlled by the control board (14), being responsible for processing the signals of the myoelectric sensors (4) and translating the signals to movement in the prosthesis, when triggering a command with the myoelectric sensors (4), the control board (14) is responsible for sending the corresponding signals to each of the servo motors (12) to generate the actions in the prosthesis, servo motors (12) move a pulley which in turn entangles a cable inside the fingers which are the ones that transmit the force of the servo motors to the fingers. The distal fingers (5) are connected to the proximal fingers (6) by means of Chicago type screws (11), a mechanism for the extension of the hand is included which consists of torsional springs (10) that generate a force in the opposite direction to that of the servo motors (12), so that, when the force for bending is no longer applied, the springs return the fingers of the prosthesis to their original position. The distal fingers (5), proximal fingers (6) and the drive system (7) are held together due to the Chicago type screws (11) allowing the movement of the Hand palm module (1). The drive system comprises the servomotor (12), control board (14) and hand palm cap (13), which take care of the independent movement of the thumb and forefinger fingers, and together with the middle, ring and pinky fingers. The combination of these movements allows to program different functions that facilitate the performance of the user's day-to-day activities. The control board (14) handles the input signals of myoelectric (4) sensors, and output signals for the movement of the servo motors (12) allowing very simple control without the need for prolonged training. The Hand palm system (1) has an organic design that closely resembles a real human hand, which makes it very attractive to the human eye. According to FIG. 3, you have the Socket-Chassis module (2) that is coupled with the Hand palm module (1) by means of an assembly guide (8). This system is responsible for storing the battery (17) that supplies power to the device. The average battery life (17) is approximately 8 hours of continuous work, allowing its use during the day and performing the recharge during the night. The Socket-Chassis module (2) consists of the cover (16) for battery protection and containment (17), a jack (18) specially designed to be able to attach the battery (17) to the Socket-Chassis (2), the Jack (18) has 2 plates (19a,19b) attached which are used for coupling to the battery current terminals (17), the display (21) shows the menu of options programmed to generate the movement of the prosthesis. In the wrist area, all the connections necessary for the communication of the myoelectric sensors (4) and energy, which communicate with the control board (14) are located.

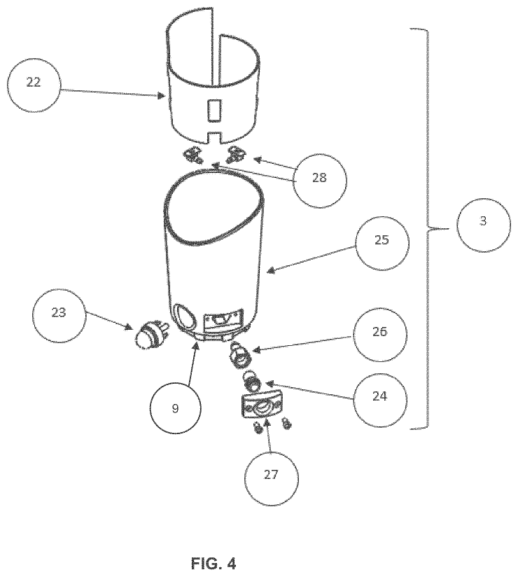

Regarding FIG. 4, the Socket-stump module (3) is coupled with the Socket-Chassis module (2), via the assembly guide (9). It is responsible for the attachment to the user's limb. It is composed of a pneumatic circuit (22) on the inside, which, when entering air volume, changes its shape, imprisoning the stump and keeping it in position, this allows to elaborate defined measurements of sizes, providing the user the one that best fits the size of his stump even if its dimensions change over time. It also comprises an air intake valve (23), this WALBRO WYJ33 type valve is used for air entry into the pneumatic circuit (22) in one direction, the bulb is pressed into the side of the stump socket (3) by pushing the air that is stored inside the bulb, into the outlet tube, which is connected by a hose (not illustrated) to the 3.2 mm (28) connector for air entry into the pneumatic circuit (22) by removing the pressure from the bulb the vacuum generated inside performs an air suction function, which fills the air bulb, this generates an air inlet cycle which is used to control the internal pressure of the pneumatic circuit and an air ejection valve (24) to release the pressure and remove the prosthesis easily.

The Socket-stump module (3) is in direct contact with the user and serves to grip your stump. It consists of an internal pneumatic circuit (22) that inflates and deflates for stump grip in a comfortable and versatile way, an air intake valve (23), a Socket-Chassis (25), a connecting coupling (26), exhaust valve cover (27) and air exhaust valve (24). By pressing the air intake valve (23), it injects press-pressure air that causes the pneumatic circuit (22) to increase its volume and thus imprison the user's stump, the exhaust valve (24) serves to release the pressure so that the prosthesis can be removed from the user's arm.

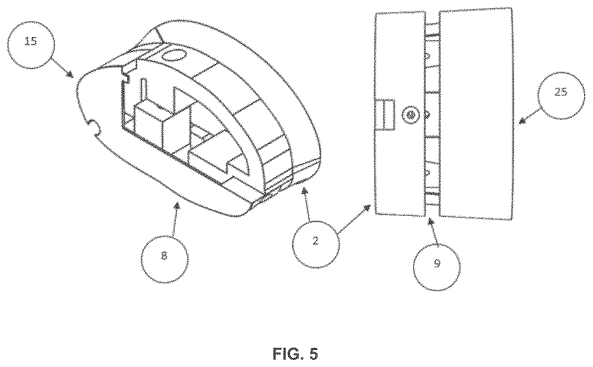

With regard to FIG. 5, you can see the assembly guides (9, 8) on the bottom of the Socket (25), which is the housing that protects the stump and functions as a chassis for the stump socket module (3) for the assembly (9) with the Socket-Chassis module (2), making a modular assembly that allows to change the modules in case of maintenance or upgrade. these guides serve as guide rails for correct positioning and bonding between components, the assembly (9) has the mode of being able to rotate certain degrees for adjustment depending on the person. The Assembly Guide (8) is designed to be assembled in one way and have a solid assembly.

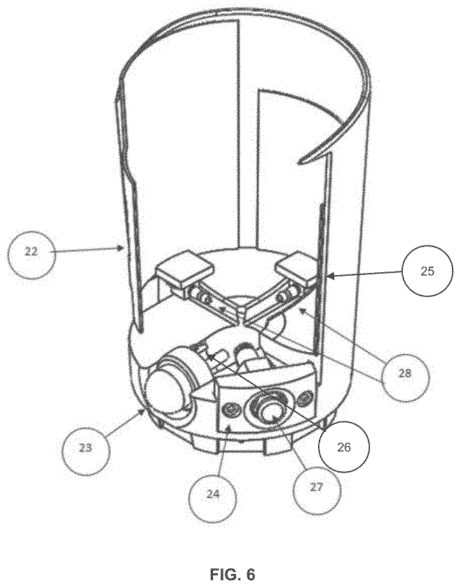

With regard to FIG. 6, a section view of the bottom of the Socket-stump module (3) can be observed to give a clearer picture on the Socket-stump (3), this comprises inside a pneumatic circuit (22) which has two connectors (28) of 3.2 mm, one connector is connected to the air intake valve (23) by means of hoses, the other 3.2 mm connector (28) is connected to a coupling (26), for the threading of the air exhaust valve (24).

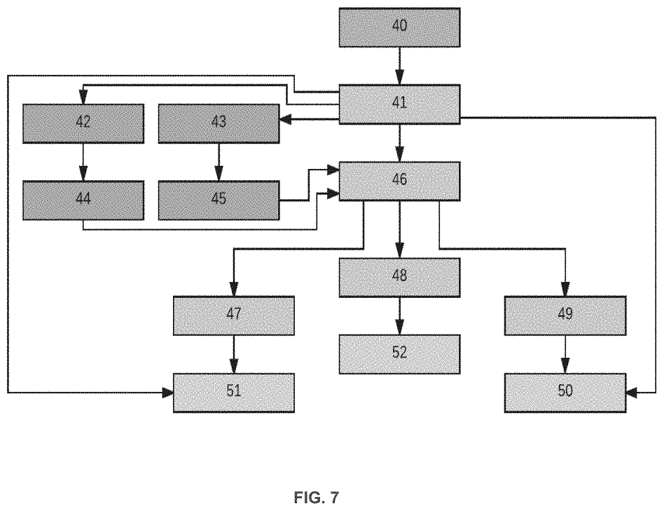

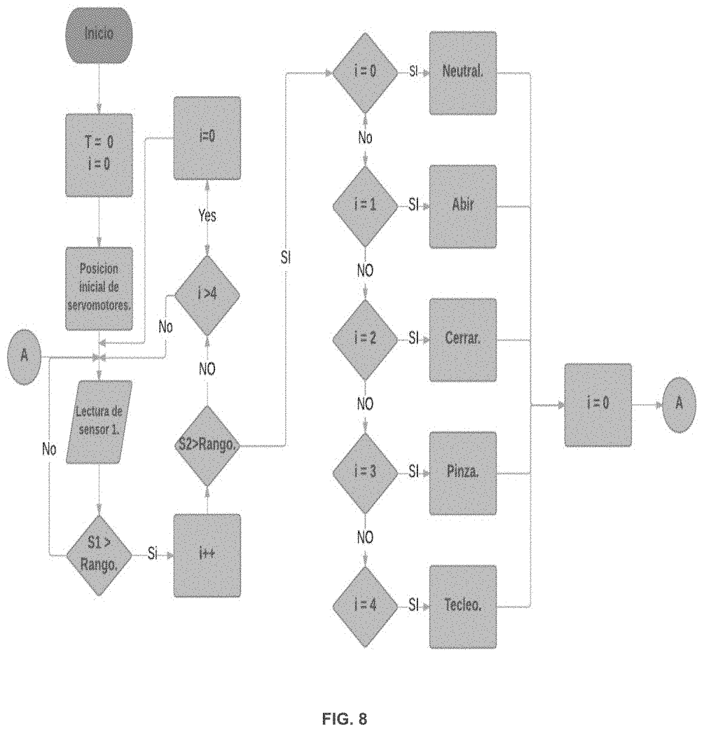

With regard to FIG. 7, a diagram of the operation of the control board (14) of the invention is shown, the energy is provided by a battery (17) which passes to a voltage regulator (41), which powers 2 myoelectric sensors (42,43), the controller (46) and the 3 servo motors (50,51,52). Myoelectric sensors (42,43) are responsible for recording and translating signals from the user's body to an analogy signal, which the controller (46) records these signals, and through a control algorithm (FIG. 8) the controller (46) sends a series of pulses (47,48,49) to each of the servo motors (50,51,52), to generate a series coordinates movement which translate into functions of the prosthesis.

About FIG. 8, a flowchart is shown which gives a representation of the operation of the control algorithm of the modular arm prosthesis. The system initializes the timer variable (T), variable (i) and places in its initial position to the modular arm prosthesis, the cycle starts with sensor reading 1, when the sensor 1 condition is met, exceeds a certain programmed range on the controller (S1>), performs a sum to the value of “i”, if the following sensor condition 2 (S2<) is not met, the continuous cycle and if the value of “i” is greater than 4, the value of “i” is reset to 0. If sensor condition 2 (S2>) is met, a function of the prosthesis is triggered, determined by the value of “i”, when the function is activated the value of “i” is reset to 0, and thus the cycle begins again (start).

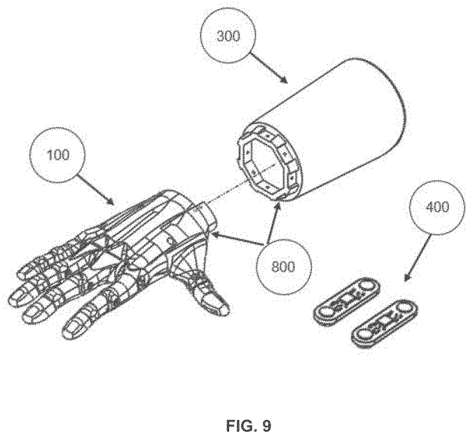

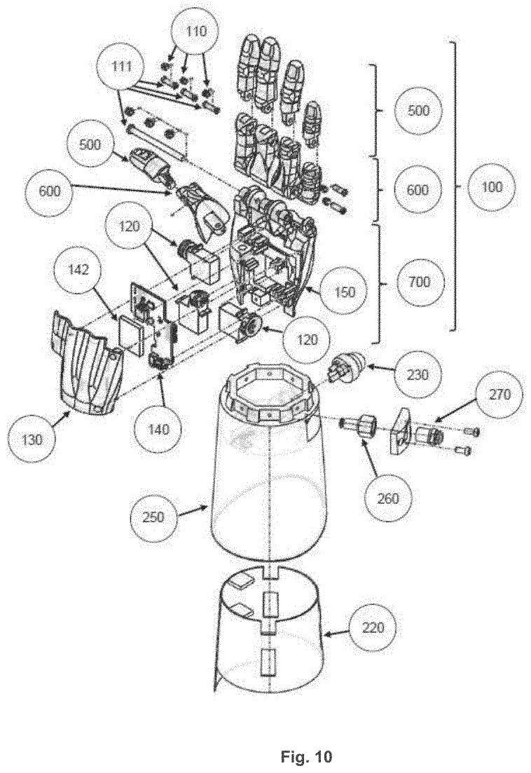

FIGS. 9 and 10 illustrate an embodiment of the invention.

With reference to FIG. 9, it can see the different parts/modules of the prosthesis with their respective myoelectric sensors (100, 300, 400). The arm prosthesis is a device for people who lack the limb at the transradial level, either due to congenital causes or amputations. The prosthesis consists of two main modules (100, 300), the first module called Hand palm (100) is responsible for storing the drive system, the control board and the mechanism responsible for the movement of the fingers and battery, and the module called Socket-stump (300) which is responsible for attaching the prosthesis to the end user, which are connected to each other by means of assembly guides (800) which allow a firm and secure assembly.

In relation to FIG. 10, the Hand palm module (100), comprises distal fingers (500) and proximal fingers (600) which are the main part for gripping objects by duplicating the basic functions of a hand that are flex, extension, and clamp. For the realization of these movements is used the drive system (700) responsible for generating the gripping force by means of servo motors (120), which are controlled by the control board (140), being responsible for processing the signals of the myoelectric sensors (400) and translating the signals in motion into the prosthesis, when triggering a command with the myoelectric sensors (400), the control board (140) is responsible for sending the corresponding signals to each of the servo motors (120) to generate the actions in the prosthesis, servo motors (120) move a pulley which in turn entangled a cable inside the fingers which are the ones that transmit the force of the servo motors to the fingers. The distal fingers (500) are connected to the proximal fingers (600) by means of Chicago type screws (111), a mechanism for the extension of the hand is included consisting of torsional springs (110) that generate a force in the opposite direction to that of the servomotors (120), so that, When you stop applying the force for bending, the springs return the fingers of the prosthesis to their original position. The distal fingers (500), proximal fingers (600) and the drive system (700) are held together thanks to the Chicago type screws (111) allowing the movement of the Hand palm module (100). The drive system comprises the servo motor (120), control board (140) and hand palm cap (130), which take care of the independent movement of the thumb and forefinger fingers, and together with the middle, ring and pinky fingers. The combination of these movements allows to program different functions that facilitate the performance of the user's day-to-day activities. The control board (140), is responsible for manipulating the input signals of myoelectric sensors (400), and output for the movement of the servo motors (120) allowing very simple control without the need for prolonged training. The Hand palm module (100) has an organic design that closely resembles a real human hand, which makes it very attractive to the human eye.

The Socket-stump module (300) is based on a pneumatic circuit (220) on the inside, which, when entering air volume, changes its shape, imprisoning the stump and keeping it in position, this allows to elaborate defined measurements of sizes, providing the user with the one that best fits the size of his stump even if its dimensions change over time. It also comprises an air intake valve (230), this model valve type WALBRO WYJ33 serves for the entry of air into the pneumatic circuit (220) in a single direction, the bulb is pressed on the side of the stump socket (300) pushing the air that is stored inside the bulb, towards the outlet tube, which is connected by a hose (not illustrated) to the 3.2 mm (280) connector for air entry into the pneumatic circuit (220) by removing the pressure from the bulb the vacuum that is generated inside performs a suction function which fills the air bulb, this generates an air inlet cycle which is used to control the internal pressure of the pneumatic circuit and an air ejection valve (240) to release the pressure and remove the prosthesis easily.

The present invention has been described and illustrated in its preferred embodiment, however, an skill person in the art will be able to visualize variations that fall within the scope of the following claims.

1. Modular arm prosthesis, comprising:
a first module including a drive system and a finger movement mechanism and a control board;
a second module that has a first side configured to attach to the first module and a second side configured to attach to a user's stump; the second module includes inside a pneumatic system with an air intake valve to inflate an inner part of the second module and an air ejection valve to deflate the inner part of the second module.

2. The modular arm prosthesis according to claim 1, further comprising an intermediate module between the first module and the second module, the intermediate module includes a battery of power supply for the prosthesis, said battery supplies power to the control board.

3. The modular arm prosthesis in accordance with claim 1, wherein the first module includes a power supply battery for the prosthesis, said battery supplies power to the control board located in the first module.

4. A module for modular arm braces, comprising:
a first side configured to attach to an additional module and a second side configured to attach to a user's stump; the module includes, inside, a pneumatic system with an air intake valve to inflate an inner part of the module and an air ejection valve to deflate the inner part of the module.

5. The module for modular arm prosthesis according to claim 4, further comprising an intermediate module between the module and the additional module, the additional module includes a motor system and a mechanism for finger movement and a control board, the intermediate module includes a battery of power supply for the prosthesis, said battery supplies power to the control board.

6. The module for modular arm prosthesis according to claim 4, wherein the additional module includes a power supply battery for the prosthesis, said battery supplies power to a control board located in the additional module.
